(19) United States
(12) Patent Application Publication
(10) Pub. No.: US 2025/0205485 A1
(43) Pub. Date: Jun. 26, 2025

(54) SPINAL CORD INJURY TREATMENT

(71) Applicant: INCUBE LABS, LLC, San Jose, CA (US)

(72) Inventor: Mir A. IMRAN, San Jose, CA (US)

(73) Assignee: INCUBE LABS, LLC, San Jose, CA (US)

(21) Appl. No.: 18/847,306

(22) PCT Filed: Mar. 14, 2023
(86) PCT No.: PCT/US2023/015160

§ 371 Date: Sep. 16, 2024

Related U.S. Application Data
(60) Provisional application No. 63/320,011, filed on Mar. 15, 2022.

(51) Int. Cl. A61N 1/36; A61N 1/08

(52) U.S. Cl. CPC A61N 1/36007; A61N 1/08; A61N 1/36139

(57) ABSTRACT

Controlling urinary function of a patient is provided. A first electrical stimulation is applied, by a controller using a pulse generator to generate electrical waveforms and one or more electrodes positioned in electrical contact with a nervous system of a patient, to relax a bladder of the patient to allow filling of the bladder and to prevent urination. Responsive to a readiness trigger being indicated to empty the bladder, the controller adjusts the operation of the pulse generator to apply a second electrical stimulation to the nervous system using the one or more electrodes to facilitate a voiding cycle of the bladder. Responsive to completion of the voiding cycle, the controller resumes applying, by readjusting the operation of the pulse generator, the first electrical stimulation to the nervous system using the one or more electrodes to again relax the bladder of the patient and to prevent urination.

### CROSS-REFERENCE TO RELATED APPLICATIONS

This application claims the benefit of U.S. provisional application Ser. No. 63/320,011 filed Mar. 15, 2022, the disclosure of which is hereby incorporated in its entirety by reference herein.

### TECHNICAL FIELD

Embodiments of the present disclosure relate to medical apparatuses and methods. More particularly, embodiments of the present disclosure relate to systems and methods for allowing paralyzed and incontinent patients to selectively control urinary function.

### BACKGROUND

Many disorders result in loss of a patient's ability to voluntarily control bladder function. Patients suffering from spinal cord injuries, for example, may lose the ability to voluntarily control urination as well as to sense a full bladder. Spinal cord injury patients may also suffer low bladder volume, which can cause urine to back up into the kidneys.

Some patients rely on chronic use of a catheter. Yet, catheters present a risk of infection which is exacerbated by the frequent need for catheter exchange. Moreover, catheters may drain into a bag, which may be undesirable to carry while the patient is traveling.

### SUMMARY

In one or more illustrative embodiments, a method for selectively controlling urinary function is provided. A first electrical stimulation is applied, by a controller using a pulse generator to generate electrical waveforms and one or more electrodes positioned in electrical contact with a nervous system of a patient, to relax a bladder of the patient to allow filling of the bladder and to prevent urination, the one or more electrodes being electrically coupled to the pulse generator to receive the electrical waveforms from the pulse generator. Responsive to a readiness trigger being indicated to empty the bladder, the controller adjusts the operation of the pulse generator to apply a second electrical stimulation to the nervous system using the one or more electrodes to facilitate a voiding cycle of the bladder. Responsive to completion of the voiding cycle, the controller resumes applying, by readjusting the operation of the pulse generator, the first electrical stimulation to the nervous system using the one or more electrodes to again relax the bladder of the patient and to prevent urination.

In one or more illustrative embodiments, a system for selectively controlling urinary function is provided. A pulse generator is connected to a nervous system of a patient by one or more electrodes configured to receive electrical waveforms from the pulse generator. A controller is programmed to apply a first electrical stimulation from the pulse generator to the nervous system to relax a bladder of the patient to allow filling of the bladder and to prevent urination. Responsive to a readiness trigger being indicated to empty the bladder, the controller is programmed to apply a second electrical stimulation from the pulse generator to facilitate a voiding cycle of the bladder. Responsive to completion of the voiding cycle, the controller is programmed to resume reapply the first electrical stimulation to again relax the bladder of the patient and to prevent urination.

### BRIEF DESCRIPTION OF THE DRAWINGS

FIG. 1 illustrates an example system for applying nerve stimulation to control urination in a patient, in accordance with an embodiment of the disclosure;

FIG. 2 illustrates a block diagram including details of a nerve stimulation device, in accordance with an embodiment of the disclosure;

FIG. 3A illustrates a block diagram of an example of a pudendal nerve implementation, in accordance with an embodiment of the disclosure;

FIG. 3B illustrates a block diagram of an alternate example of the pudendal nerve implementation, in accordance with an embodiment of the disclosure;

FIG. 4 illustrates a block diagram of an example of the sacral nerve implementation, in accordance with an embodiment of the disclosure;

FIG. 5 illustrates an example process for the applying nerve stimulation to control urination in a patient, in accordance with an embodiment of the disclosure; and

FIG. 6 illustrate an example process for closed loop adjustment of the nerve stimulation to control urination in a patient.

### DETAILED DESCRIPTION

As required, detailed embodiments of the present disclosure are disclosed herein; however, it is to be understood that the disclosed embodiments are merely exemplary of the disclosure that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

The term “in an embodiment” or a variation thereof (e.g., “in another embodiment”, “in a particular example of an embodiment”, “in one embodiment”, “in an example”, or “in one or more examples,”) refers herein to use in one or more embodiments, and in no case limits the scope of the present disclosure to only the embodiment as illustrated and/or described. Accordingly, a component or feature illustrated and/or described herein with respect to one embodiment can be omitted or can be used in another embodiment (e.g., in another embodiment illustrated and described herein, or in another embodiment within the scope of the present disclosure and not illustrated and/or not described herein). As used herein, the terms “comprising”, “comprise”, “comprises”, “includes”, and “including” are intended to mean that the compositions and methods include the recited elements, but do not exclude others.

Embodiments of the present disclosure provide improved systems and methods for controlling urination in patients unable to voluntarily control urination. Systems according to the present disclosure include an implantable nerve stimulation device having a controller, a pulse generator configured to provide current in the form of an electrical waveform, and electrodes implanted into the patient configured to deliver the current to the patient's nerves. A remote control may communicate with the nerve stimulation device to receive alerts and send commands to the nerve stimulation device. This remote control may allow the patient or a caregiver to send commands to the controller of the nerve stimulation device to invoke a voiding cycle of the bladder. As used herein, the term “nerve stimulation” refers to applying electrical stimulation to a nerve, whether the stimulation is intended to cause or block afferent or efferent nurse pulses.

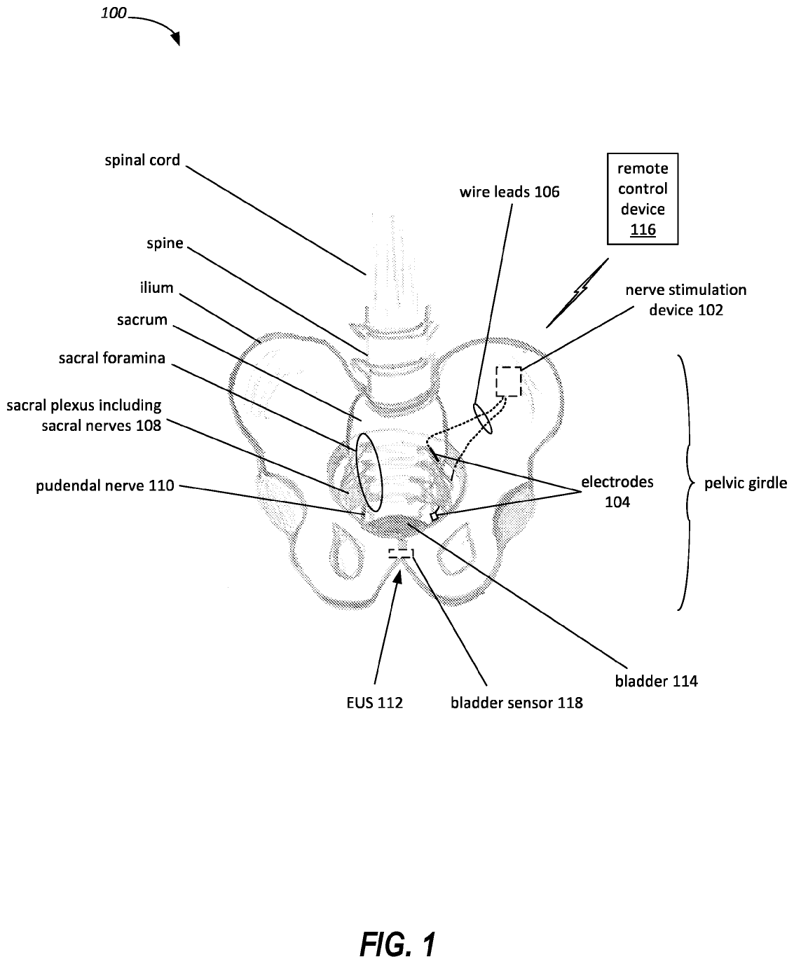

FIG. 1 illustrates an example system 100 for applying nerve stimulation to control urination in a patient. The system 100 includes a nerve stimulation device 102 connected to control electrodes 104 via wire leads 106. The electrodes 104 are placed adjacent or around an applicable area or feature to be stimulated, and the wire leads 106 are routed in a suitable path or paths between the nerve stimulation device 102 and the electrodes 104. By way of example, in the embodiment illustrated in FIG. 1, a nerve stimulation device 102 is positioned in an area of the buttocks and two wire leads 106 are routed to respective electrodes 104. A first electrode 104a is illustrated as being routed through the S1 foramen and positioned adjacent a sacral nerve in the sacral plexus. A second electrode 104b is illustrated as being routed along a posterior portion of the ilium to a cuff electrode positioned around the left pudendal nerve 110. Many other combinations of wire leads 106 and electrodes 104, and the placement and routing of them, are contemplated by the present disclosure. For example, the nerve stimulation device 102 may be implanted in an area of the buttocks or pelvic girdle, or in another area having a cavity suitable for implantation of the nerve stimulation device 102. The nerve stimulation device 102, electrodes 104, and wire leads 106 may be implanted into the patient to provide signals to sacral nerves 108, pudendal nerves 110 and/or external urethral sphincter (EUS) 112 of the patient to control the patient's bladder 114. The nerve stimulation device 102 may be configured to wirelessly communicate with a remote-control device 116 external to the patient.

In some embodiments, a bladder sensor 118 may optionally be implanted in the patient. For example, the bladder sensor 118 may be implanted between the pubic bone and the bladder such that the bladder rests on the bladder sensor 118. The bladder sensor 118 (or an additional bladder sensor 118) may be positioned on or in the bladder 114. The bladder sensor 118 may include a weight sensor to measure the weight of the bladder. The bladder sensor 118 may additionally or alternatively measure pressure, bladder wall density, bladder wall opacity, or other indication of fullness of the bladder 114. The bladder sensor 118 may additionally include a gyroscope and/or an accelerometer to aid in determining the orientation of the patient. This may be useful as the measurements from the bladder sensor 118 may depend on the orientation of the patient (e.g., the weight may be sensed differently when the patient is in a position where the bladder sensor 118 is below the bladder, such as when the patient is in a sitting or standing position, than when the bladder sensor 118 is not below the bladder, such as when the patient is in a prone or supine position). The bladder sensor 118 may be in communication with the nerve stimulation device 102 or the remote-control device 116 to provide information to the nerve stimulation device 102 or the remote-control device 116 with respect to the filled status of the bladder 114.

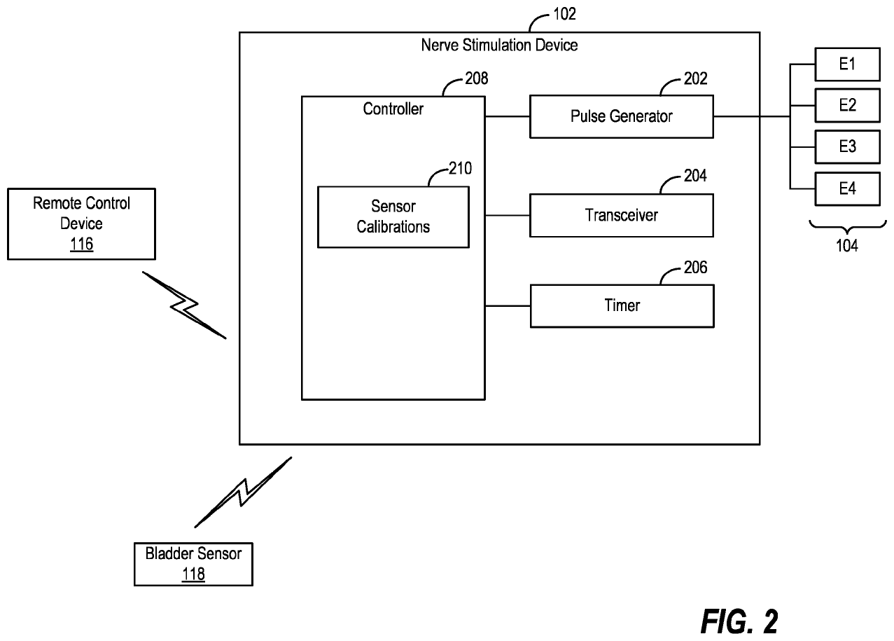

FIG. 2 illustrates further details of the nerve stimulation device 102. As shown, the nerve stimulation device 102 includes components such as a pulse generator 202, a transceiver 204, a timer 206, and a controller 208. It should be noted that the example of FIG. 2 is not limiting, and nerve stimulation devices 102 having more, fewer, or different components may be utilized.

The pulse generator 202 may be configured to provide signals to one or more outputs, where each of the outputs is connected to one or more of the electrodes 104 via wire leads 106. The signals may be activated or deactivated under the operation of the controller 208. The pulse generator 202 may also be configured to allow one or more characteristics (e.g., frequency, voltage, current) of the signals to be controllable by the controller 208.

The transceiver 204 may be configured to receive wireless signals from the remote-control device 116. In an example, the transceiver 204 may allow the controller 208 to communicate with the remote-control device 116 over Wi-Fi or BLUETOOTH or another standard, or a proprietary, communication protocol. In another example, the transceiver 204 may allow the controller 208 to receive data from and/or communicate with the implantable bladder sensor 118.

The timer 206 may be a type of clock used for measuring specific time intervals. In some examples timer 206 may be implemented as hardware while in other examples the timer 206 may be implemented in software. The timer 206 may have a timeout period settable by the controller 208 and may be configured to raise a signal to the controller 208 responsive to occurrence of the timeout period.

The controller 208 may be programmed to control function of the bladder 114 of the patient by directing the pulse generator 202 to send signals of predefined characteristics to one or more sacral nerves 108 and/or one or more pudendal nerves 110 and/or the EUS 112 via the electrodes 104. The signals may have one or more characteristics (e.g., frequency, voltage, current) configured to stimulate and/or block the patient's nerve signals to cause a physiological action involved in the urination process. Such actions may include opening of the EUS 112, contraction of the bladder 114, or relaxation of the bladder 114. Frequency may be a useful characteristic for producing a specific physiologic action. For example, high frequencies may be used to cause relaxation or opening of the EUS 112 (e.g., 5-10 kHz), while lower frequencies may be used to contract the bladder 114 (e.g., 20-100 Hz) and very low frequencies may be used to relax the bladder 114 (e.g., 2-20 Hz).

The electrodes 104 may be monopolar, bipolar, tripolar, or even higher order polar electrodes 104. In some examples, the stimulation may be delivered through monopolar electrodes 104. In other examples, the electrodes 104 may be multipolar electrodes 104 to minimize the amount of stimulation effect to other nerves or nerve portions. For instance, bipolar electrodes 104 may be used to avoid the passage of current through the nerve to focus stimulation on a small volume. In another example, tripolar electrodes 104 having an additional guard band may be used to further focus the stimulation. In one example, the tripolar configuration may include concentric poles with a plus minus plus (+−+) polarity, while in another example the tripolar configuration may include concentric poles with minus plus minus (−+−) polarity.

In some examples, the electrodes 104 may be combined into wire leads 106 having a plurality of electrodes 104. For instance, a wire lead 106 may be installed that includes multiple electrodes 104 or multiple pairs of electrodes 104. In one non-limiting example, a wire lead 106 may include two or more bifold pairs of electrodes 104. This may allow a single installation of a wire lead 106 to include multiple redundant electrodes 104. If an issue occurs with a portion of the electrodes 104 of the wire lead 106, then the controller 208 may be able to switch to use of other electrodes 104 of the wire lead 106 without requiring the patient to undergo a further surgical procedure.

The controller 208 may further utilize the implantable bladder sensor 118 to sense information corresponding to a degree of filling of the patient's bladder 114. In an example, the bladder sensor 118 may be a pressure sensor configured to measure or sense indicia of weight or pressure. In another example, the bladder sensor 118 may be a sensor adapted to measure or sense indicia of one or more of bladder wall stretch, bladder distension, or the like. A bladder full condition may be determined based on the measured or sensed indicia, by the bladder sensor 118 and/or the controller 208. The bladder full condition may include one or more of a bladder volume exceeding a predefined threshold, a bladder pressure exceeding a predefined threshold, and/or a bladder weight exceeding a predefined threshold. Such thresholds may be adapted based on a position and/or movement of the patient.

The nerve stimulation device 102 may be installed to the patient in a pudendal configuration or in a sacral configuration. As compared to one another, the pudendal and sacral implementations may utilize different quantities and placement of the electrodes 104 as well as different frequency ranges for controlling function of the bladder 114 of the patient.

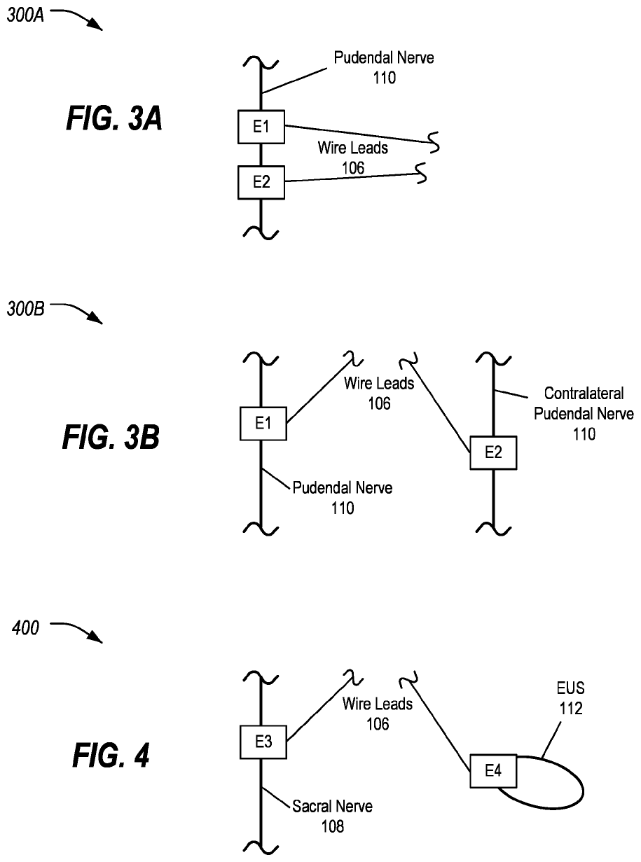

FIG. 3A illustrates an example 300A of a pudendal nerve 110 configuration. In the example pudendal nerve 110 configuration, a first electrode E1 and a second electrode E2 may each be placed on or adjacent to a pudendal nerve 110 (either the left or right pudendal nerve). In an embodiment, the first electrode E1 may be operable to apply 5-10 Hz stimulation or 20-100 Hz stimulation to the pudendal nerve 110 to effectuate bladder relaxation and contraction, respectively, and the second electrode E2 may be operable to apply 5-10 kHz stimulation to the pudendal nerve 110 to effectuate relaxation of the EUS 112. Other frequencies outside these example ranges may also be used.

FIG. 3B illustrates an alternate example 300B of the pudendal nerve configuration. In the alternate example pudendal nerve configuration, the first electrode E1 may be placed on or adjacent to a first pudendal nerve 110 (left or right pudendal nerve) and the second electrode E2 may be placed on or adjacent to the contralateral pudendal nerve 110. Optionally, a second instance of the second electrode E2 may be placed on or adjacent to the first pudendal nerve 110, such as to increase a capability of the system 100 to effectuate relaxation of the EUS 112. As with the example 300A, in the alternate example 300B the first electrode E1 may be operable to apply 5-10 Hz stimulation or 20-100 Hz stimulation, and the second electrode(s) E2 may be operable to apply 5-10 kHz stimulation. Other frequencies outside these example ranges may also be used.

FIG. 4 illustrates an example 400 of a sacral nerve 108 configuration. In the example sacral nerve 108 configuration, a sacral electrode E3 may be placed on or adjacent to the sacral nerve 108 and an EUS electrode E4 may be placed on or adjacent to the EUS 112. The sacral electrode E3 may be operable to apply 5-20 Hz stimulation or 20-100 Hz stimulation to the sacral nerve 108. The EUS electrode E4 may be operable to apply 5-20 kHz stimulation to the EUS 112. Other frequencies outside these example ranges may also be used. Two EUS electrodes E4 (e.g., at a 60 degree, 90 degree, or 180 degree distance from each other around a periphery of the EUS 112) may be used such as to increase a capability of the system 100 to effectuate relaxation of the EUS 112.

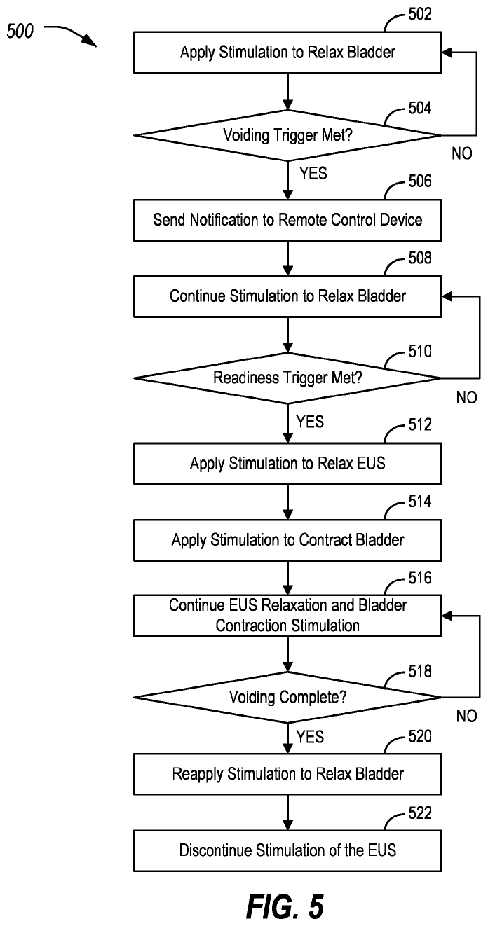

FIG. 5 illustrates an example process 500 for applying nerve stimulation to control urination in a patient. In an example, the process 500 may be performed under the control of the controller 208 by the system 100 configured in a pudendal or sacral configuration. Generally, the process 500 may initiate in a resting state. In the resting state, the controller 208 applies a first electrical current pattern from the pulse generator 202 to the nervous system of the patient to relax the bladder 114 of the patient. In certain patient populations such as in spinal cord injury (SCI) patients, the bladder 114 may be generally in a contracted state which allows a small volume of urine in the bladder and as such, if the bladder 114 is not relieved frequently or continuously, the urine may be forced into the kidneys and cause renal failure. In these and other patient populations, relaxation of the bladder 114 may allow greater capacity of the bladder 114 (which capacity may also increase over time with continued applied nerve stimulation), and in some patients may prevent involuntary urination. Responsive to receipt of a command to empty the bladder 114, the controller 208 applies a second electrical current pattern waveform from the pulse generator 202 to facilitate a voiding cycle of the bladder 114. Responsive to completion of the voiding cycle, the controller 208 may be programmed to reapply the first electrical current pattern to again relax the bladder 114.

More specifically, at operation 502, the controller 208 applies nerve stimulation to relax the bladder 114. This relaxation stimulation may be applied to increase bladder volume by relaxing the bladder 114 from its normally contracted state. In an example pudendal configuration such as shown in FIGS. 3A-3B, the relaxation stimulation may be accomplished by the controller 208 applying 5-10 Hz stimulation to the pudendal nerve via electrode E1. In an example sacral configuration such as shown in FIG. 4, the relaxation stimulation may be accomplished by applying 5-20 Hz stimulation to the sacral nerve via electrode E3.

At operation 504, the controller 208 determines whether a voiding trigger has been met. Voiding may be indicated based on various conditions. In an example, the periodic timer 206 may be used to determine when urination should be performed, such as at expiration of a defined time period. In some examples, the period of the timer 206 may vary for wake vs. sleep cycles of the patient. In another example, the voiding trigger may be met responsive to a signal from the bladder sensor 118 indicating a full condition of the bladder 114 or providing indicia which the controller 208 then determines constitutes a voiding trigger.

At operation 506, upon the voiding trigger being met, the controller 208 sends a notification to the remote-control device 116. The notification may be used to indicate to the patient or a caretaker that voiding of the bladder 114 is indicated.

At operation 508, the controller 208 continues stimulation to relax the bladder 114. In an example, the stimulation being applied at operation 502 continues despite the determination of a voiding trigger having been met and the notification having been sent to the remote-control device 116.

At operation 510, the controller 208 determines whether a readiness trigger condition has been met. The resting state may be maintained by the controller 208 until the readiness trigger condition is indicated. In an example, the notification sent to the remote-control device 116 at operation 506 may be acknowledged by the patient or caregiver and a command (which may be the acknowledgment itself) may be entered into the remote-control device 116 to initiate the void cycle. In such a case, a readiness trigger may be sent to the controller 208 from the remote-control device 116 to indicate to the controller 208 that the patient is prepared for urination. In another example, the controller 208 may receive a signal from a urinal sensor indicating that a urinal is detected (e.g., via the transceiver 204), where the signal from the urinal sensor is interpreted by the controller 208 as the readiness trigger condition.

Responsive to readiness for urination being indicated (at operation 510), the voiding cycle may be performed. This may generally include the controller 208 applying stimulation to the EUS 112 to relax the EUS 112, as well as applying additional stimulation to contract the bladder 114.

More specifically, at operation 512, the controller 208 applies stimulation to relax the EUS 112. In an example pudendal configuration, the EUS-relaxing stimulation may be accomplished by the controller 208 applying 5-10 kHz stimulation to the pudendal nerve (e.g., via the electrode E2 in FIG. 3A or FIG. 3B). In an example sacral configuration, the EUS-relaxing stimulation may be accomplished by the controller 208 applying 5-20 kHz stimulation to the EUS 112 (e.g., via the electrode E4 in FIG. 4).

At operation 514, the controller 208 applies stimulation to contract the bladder 114. In an example pudendal configuration, the bladder contracting stimulation may be accomplished by the controller 208 applying 20-100 Hz stimulation to the branch of the pudendal nerve 110 (e.g., via the electrode E1 in FIG. 3A or FIG. 3B). In an example sacral nerve configuration, the bladder contracting stimulation may be accomplished by the controller 208 applying 20-100 Hz stimulation to the sacral nerve 108 (e.g., via the electrode E3 in FIG. 4). In either a pudendal or a sacral implementation, a single electrode may be used to apply both the stimulation for bladder 114 relaxation at operation 502 and stimulation for bladder 114 contraction at operation 514, although separate electrodes may be used for operation 502 and operation 514 instead.

At operation 516, the controller 208 continues to apply the EUS-relaxation stimulation and bladder-contraction stimulation. The EUS-relaxing stimulation and the bladder-contracting stimulation may accordingly be applied until the voiding is complete.

At operation 518, the controller 208 determines whether voiding of the bladder 114 is complete. In an example, a bladder empty condition may be detected responsive to user input to the remote-control device 116. In another example, the bladder empty condition may be detected responsive to a urinal weight reaching a predefined threshold, such as via a urinal sensor or scale configured to provide weight information to the remote-control device 116. In yet another example, the bladder empty condition may be detected responsive to a urinal fill level reaching a predefined threshold, such as via a urinal sensor or other sensor configured to provide level information to the remote-control device 116. In still another example, the bladder empty condition may be detected responsive to expiration of the predefined timer 206 (e.g., a one-minute interval, a two-minute interval).

As some further examples, a bladder empty condition may be detected by the bladder sensor 118 to indicate when the voiding is complete. The bladder empty condition may include one or more of a bladder volume being below a predefined empty threshold, a bladder pressure being below a predefined empty threshold, and/or a bladder weight being below a predefined empty threshold. In another example, the voiding being complete may additionally or alternatively be determined considering past history of emptying for the detected bladder volume, weight, and/or pressure, where the past history is determined from entries made into the remote device by the patient or caregiver, and/or learned by the algorithm over time based on, e.g., time of day, movement of patient, position of patient, such as in terms of average percent reduction in bladder volume, weight, or pressure. In some embodiments, a combination of information (e.g., information related to meeting or crossing one or more thresholds and/or historical information) may be used to determine a bladder empty condition.

Responsive to the voiding being complete, the stimulation to relax the EUS 112 may be discontinued to allow the EUS 112 to contract. Additionally, the stimulation to contract the bladder 114 may be discontinued and the stimulation to relax the bladder 114 may be resumed. At operation 520, the controller 208 reapplies the stimulation applied at operation 502 to relax the bladder 114 and discontinues applying the stimulation to contract the bladder 114 previously being applied from operation 514. At operation 522, the controller 208 discontinues the EUS-relaxation stimulation previously being applied as described for operation 512. Thus, after operation 522, the process 500 has returned to the resting state (e.g., at operation 502). Thus, by using the implantable nerve stimulation device 102, nerve stimulation is used to control urination in a patient.

In embodiments including the bladder sensor 118, the controller 208 may be further programmed to compute an effectiveness measure. For instance, the controller 208 may record aspects of the fill level of the bladder 114 before and after urination. These aspects may include, for example, pressure, volume, or weight of the bladder 114. Using the recorded aspects, the controller 208 may compute an effectiveness of the voiding of the bladder. In one example, the effectiveness may be computed as a ratio of the aspects of the fill level before the voiding divided by the same aspects of the fill level after the voiding.

In another variation, the controller 208 may be programmed to perform a calibration using the bladder sensor 118 as indicated by sensor calibrations 210 in FIG. 2. For instance, one or more varying predefined amounts of liquid (e.g., saline) may be introduced into the bladder 114 to allow the controller 208 to learn the relationship between each predefined amount of liquid and the readings from the bladder sensor 118 at that level of fill. These sensor calibrations 210 may be taken at different positions of the patient (e.g., supine, prone, lateral, reclined, sitting, upright) to allow for more accurate bladder fullness determination based on the position of the patient. In later operation, the controller 208 may receive position and/or motion information from a sensor worn by or implanted in the patient (e.g., a sensor including a gyroscope and/or an accelerometer), and based on the position and/or motion information utilize a position detection algorithm to determine which of the positions the patient is in. The controller 208 may use an appropriate sensor calibration 210 for the patient's position and/or motion to interpret the bladder sensor 118 readings by applying the appropriate sensor calibration 210 parameters to the present position. For example, the calibration may be used to determine the full state of the bladder 114 at operation 504 and/or the voided state of the bladder 114 at operation 518.

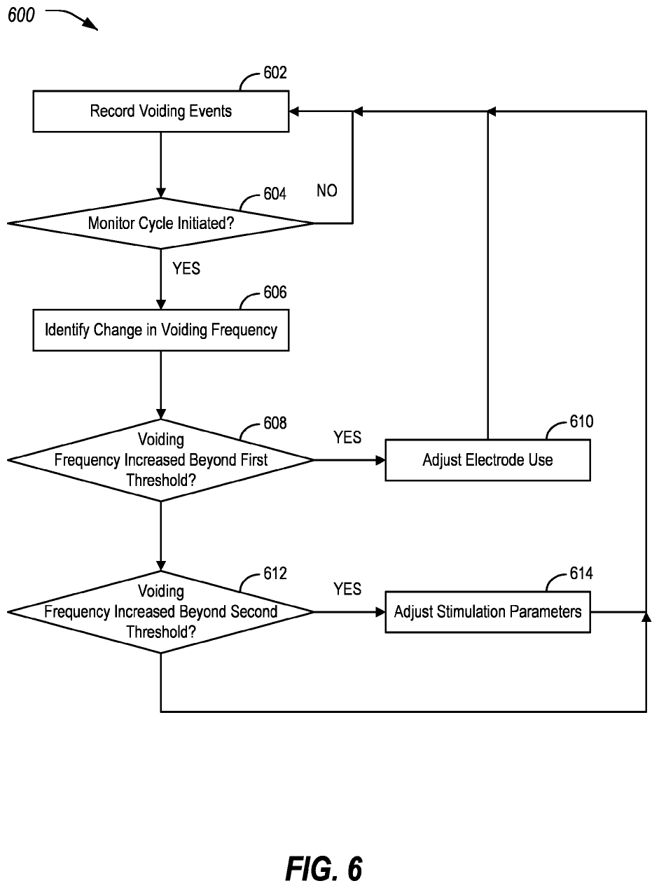

FIG. 6 illustrate an example process 600 for closed loop adjustment of the nerve stimulation to control urination in a patient. In an example, the process 600 may be performed under the control of the controller 208 by the system 100 configured in a pudendal or sacral configuration.

At operation 602, the controller 208 records voiding events that occur through use of the system 100. In an example, the controller 208 may record to the storage of the controller 208 the time at which voiding of the bladder is performed (e.g., according to operations 512-518 of the process 500). This information may be used to allow the controller 208 to compute the frequency of voiding events over time, as well as change in the frequency of the voiding events over time.

At operation 604, the controller 208 determines whether a monitor cycle is initiated. In an example, the controller 208 may be configured to periodically monitor the cycle time of the voiding events. In an example, the period may be daily, every two days, every three days, etc., but this is one example and longer or shorter periods may be used. If a monitor cycle is initiated, control proceeds to operation 606. Otherwise, control returns to operation 602 to continue recording of the voiding events.

At operation 606, the controller 208 identifies a change in the voiding frequency. For instance, the controller 208 may count the frequency of voiding events collected at operation 602 for the most recent period of monitoring from operation 604. The controller 208 may compare this frequent to a rolling average of the frequency of voiding events over time (e.g., daily, every two days, every three days). The difference between those frequencies may be used to determine whether the voiding the frequency has increased, and if so, by how much. It should be noted that this is one example approach to determining the change in the voiding frequency and different approaches may be used.

At operation 608, the controller 208 determines whether the voiding frequency exceeds a first threshold. For instance, the first threshold may be a threshold indicative of a relatively large change in the voiding frequency (e.g., two times as many voidings, five times as many voidings, ten times as many voidings, etc.). The first threshold may be indicative of an electrode 104 lead position having moved, a damaged electrode 104, or another issue with the electrodes 104 used to perform the nerve stimulation.

At operation 610, the controller 208 adjusts use of the electrodes 104 by the controller 208. For instance, the controller 208 may switch from use of the current electrodes 104 of a wire lead 106 implanted into the patient to use of other electrodes 104 of the same wire lead 106. In an example, an implanted wire lead 106 may include multiple bifold pairs of electrodes 104, and the controller 208 may switch from use of the currently used pair to a different pair of electrodes 104 in an attempt to improve performance of the system 100. After operation 610, control returns to operation 602.

At operation 612, the controller 208 determines whether the voiding frequency exceeds a second threshold. The second threshold may be a smaller different than the first threshold (e.g., 20% more voidings, 40% more voidings, 50% more voidings, etc.) The second threshold may be indicative of scarring fibrosis at the electrode 104 site or another more gradual change in the operation of the system 100.

At operation 614, the controller 208 adjusts aspects of the electrical waveforms used by the controller 208. In an example, the controller 208 may adjust the frequency, amplitude, pulse width, and/or other aspects of the electrical waveforms produced by the pulse generator 202 in an attempt to improve the operation of the system 100.

In an example, the controller 208 may be programmed with a decision tree indicative of which aspects of the electrical waveforms should be adjusted. For instance, the decision tree may indicate that the controller 208 should first attempt to increase the amplitude, and if that does not increase the voiding frequency over time, then as a next attempt operate to increase or decrease the frequency of the electrical waveforms. Or, the decision tree may instead indicate to first adjust the frequency, and if that does not increase the voiding frequency over time, then, as a next attempt, operate to increase the amplitude.

The decision tree may be determined empirically from experience with patients. In another example, the controller 208 may be configured to upload historical data descriptive of the operation of the controller 208 to a remote server. The historical data may include the voiding events as monitored at operation 602 as well as the aspects of the electrical waveforms that were adjusted at operation 614. Based on the historical data, the remote server may identify trends with respect to which adjustments to the electrical waveforms were effective. This information may be encoded in the decision tree, which may be downloaded or otherwise applied to the controller 208 to improve operation of the controller in the adjustment of the nerve stimulation to control urination in a patient.

After operation 614, control returns to operation 602.

Embodiments include without limitation the following:

In an aspect, a method for selectively controlling urinary function is provided. A first electrical stimulation is applied, by a controller using a pulse generator to generate electrical waveforms and one or more electrodes positioned in electrical contact with a nervous system of a patient, to relax a bladder of the patient to allow filling of the bladder and to prevent urination. Responsive to a readiness trigger being indicated to empty the bladder, the controller adjusts the operation of the pulse generator to apply a second electrical stimulation to the nervous system using the one or more electrodes to facilitate a voiding cycle of the bladder. Responsive to completion of the voiding cycle, the controller resumes applying, by readjusting the operation of the pulse generator, the first electrical stimulation to the nervous system using the one or more electrodes to again relax the bladder of the patient and to prevent urination.

In an aspect, the first electrical stimulation includes applying 5-10 Hz stimulation to the pudendal nerve to relax the bladder, and the second electrical stimulation includes applying 5-10 kHz stimulation to the pudendal nerve to relax the EUS and applying 20-100 Hz stimulation to the pudendal nerve to contract the bladder.

In an aspect, the first electrical stimulation includes applying 5-20 Hz stimulation to the sacral nerve to relax the bladder, and the second electrical stimulation includes applying 5-20 kHz stimulation to the EUS to relax the EUS and applying 20-100 Hz stimulation to the sacral nerve contract the bladder.

In an aspect, a notification is sent to a remote-control device responsive to a voiding trigger being met; and the readiness trigger is received from the remote-control device confirming readiness of the patient to empty the bladder.

In an aspect, the voiding trigger is indicated responsive to expiration of a timer. In some examples, the voiding trigger is indicated responsive to a bladder sensor indicating a full condition of the bladder.

In an aspect, a calibration of the bladder sensor is performed by measuring signals from the bladder sensor indicative of the level of fill of the bladder responsive to introducing one or more predefined amounts of liquid into the bladder. Signals are received from the bladder sensor, and the calibration is used to determine that the signals from the bladder sensor indicate the full condition.

In an aspect, the readiness trigger is indicated responsive to a signal from a urinal sensor indicating that a urinal is detected. In an aspect, the completion of the voiding cycle is indicated responsive to user input to the remote-control device. In an aspect, the completion of the voiding cycle is detected responsive to a urinal weight reaching a predefined threshold weight. In an aspect, the completion of the voiding cycle is detected responsive to a urinal fill level reaching a predefined threshold fill level. In an aspect, the completion of the voiding cycle is detected responsive to expiration of a predefined timeout period for urination.

In an aspect, aspects of a fill level of the bladder are recorded before and after the voiding cycle. An effectiveness of the voiding cycle is computed as a ratio of the aspects of the fill level before the voiding divided by the same aspects of the fill level after the voiding.

In an aspect, times at which voiding cycles of the bladder are performed is recorded by the controller; and responsive to the controller determining a change in frequency of the voiding cycles being greater than a first predefined threshold amount, use of the one or more electrodes is switched to use of one or more other electrodes. In an aspect the first predefined threshold amount is indicative of a changed electrode lead position.

In an aspect, responsive to the controller determining the change in the frequency of the voiding cycles is less that than the first predefined threshold amount but greater than a second predefined threshold amount, aspects of the electrical waveforms being used by the controller to the voiding cycles are adjusted. In an aspect, the second predefined threshold amount is indicative of scarring fibrosis at a nerve site of the one or more electrodes. In an aspect, the aspects of the electrical waveforms include one or more of frequency, amplitude, or pulse width. In an aspect a decision tree is utilized to identify which of the aspects of electrical waveforms are to be adjusted.

In an aspect, a system for selectively controlling urinary function is provided. A pulse generator is connected to a nervous system of a patient by one or more electrodes. A controller is programmed to apply a first electrical stimulation from the pulse generator to the nervous system to relax a bladder of the patient to allow filling of the bladder and to prevent urination; responsive to a readiness trigger being indicated to empty the bladder, apply a second electrical stimulation from the pulse generator to facilitate a voiding cycle of the bladder; and responsive to completion of the voiding cycle, resume reapply the first electrical stimulation to again relax the bladder of the patient and to prevent urination.

In an aspect, the one or more electrodes includes a first electrode connected to the pudendal nerve and a second electrode connected to the pudendal nerve, and the controller is further programmed to direct the pulse generator, as the first electrical stimulation, to apply 5-10 Hz stimulation to the pudendal nerve via the first electrode to relax the bladder; and direct the pulse generator, as the second electrical stimulation, to apply 5-10 kHz stimulation to the pudendal nerve via the second electrode to relax the EUS and to apply 20-100 Hz stimulation to the pudendal nerve via the first electrode to contract the bladder.

In an aspect, the one or more electrodes includes a first electrode connected to the sacral nerve and a second electrode connected to the EUS, and the controller is further programmed to direct the pulse generator, as the first electrical stimulation, to apply 5-20 Hz stimulation to the sacral nerve via the first electrode to relax the bladder; and direct the pulse generator, as the second electrical stimulation, to apply 5-20 kHz stimulation to the EUS via the second electrode to relax the EUS and to apply 20-100 Hz stimulation to the sacral nerve via the first electrode to contract the bladder.

In an aspect, the controller is further programmed to send a notification to a remote-control device, via a wireless transceiver, responsive to a voiding trigger being met; and receive the readiness trigger from the remote-control device, via the wireless transceiver, responsive to the remote-control device confirming readiness of the patient to empty the bladder.

In an aspect, the controller is further programmed to indicate the voiding trigger responsive to expiration of a timer. In an aspect, the system further includes a bladder sensor configured to provide a signal indicative of a fill level of the bladder to the controller, wherein the controller is further programmed to indicate the voiding trigger responsive to a bladder sensor indicating a full condition of the bladder.

In an aspect, the controller is further programmed to perform a calibration of the bladder sensor by measuring signals from the bladder sensor indicative of the level of fill of the bladder responsive to introducing one or more predefined amounts of liquid into the bladder; receive signals from the bladder sensor; and utilize the calibration to determine that the signals from the bladder sensor indicate the full condition.

In an aspect, the controller is further programmed to indicate the readiness trigger responsive to a signal from a urinal sensor indicating that a urinal is detected. In an aspect, the controller is further programmed to indicate the completion of the voiding cycle responsive to user input to the remote-control device. In an aspect, the controller is further programmed to indicate the completion of the voiding cycle responsive to a urinal weight reaching a predefined threshold weight. In an aspect, the controller is further programmed to indicate the completion of the voiding cycle responsive to a urinal fill level reaching a predefined threshold fill level. In an aspect, the controller is further programmed to indicate the completion of the voiding cycle responsive to expiration of a predefined timeout period for urination.

In an aspect, the controller is further programmed to record aspects of a fill level of the bladder before and after the voiding cycle; and compute an effectiveness of the voiding cycle as a ratio of the aspects of the fill level before the voiding divided by the same aspects of the fill level after the voiding.

In an aspect, the controller is further programmed to record times at which voiding cycles of the bladder are performed; and responsive to the controller determining a change in frequency of the voiding cycles being greater than a first predefined threshold amount, switch from use of the one or more electrodes to use of one or more other electrodes. In an aspect, the first predefined threshold amount is indicative of a changed electrode lead position.

In an aspect, responsive to the controller determining the change in the frequency of the voiding cycles is less that than the first predefined threshold amount but greater than a second predefined threshold amount, the controller adjusts aspects of the electrical waveforms being used by the controller to the voiding cycles. In an aspect, the second predefined threshold amount is indicative of scarring fibrosis at a nerve site of the one or more electrodes. In an aspect, the aspects of the electrical waveforms include one or more of frequency, amplitude, or pulse width. In an aspect the controller utilities a decision tree to identify which of the aspects of electrical waveforms are to be adjusted.

Computing devices described herein, including but not limited to the controller 208 and remote-control device 116, generally include computer-executable instructions where the instructions may be executable by one or more computing devices such as those listed above. Computer-executable instructions, such as those executed by the controller 208 and the remote-control device 116, may be compiled or interpreted from computer programs created using a variety of programming languages and/or technologies, including, without limitation, and either alone or in combination, Java™, C, C++, C#, Visual Basic, JavaScript, Python, JavaScript, Perl, etc. In general, a processor (e.g., a microprocessor) receives instructions (e.g., from a memory, a computer-readable medium, etc.), and executes these instructions, thereby performing one or more processes, including one or more of the processes described herein. Such instructions and other data may be stored and transmitted using a variety of computer-readable media.

With regard to the processes (e.g., methods, heuristics, etc.) described herein, it should be understood that, although the steps have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The abstract of the disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

While exemplary embodiments are described above, it is not intended that these embodiments describe all possible forms of the disclosure. Rather, the words used in the specification are words of description rather than limitation, and it is understood that various changes may be made without departing from the spirit and scope of the disclosure. Additionally, the features of various implementing embodiments may be combined to form further embodiments of the disclosure.

What is claimed is:

1. A method for selectively controlling urinary function, comprising:
applying, by a controller using a pulse generator to generate electrical waveforms and one or more electrodes positioned in electrical contact with a nervous system of a patient, a first electrical stimulation to relax a bladder of the patient to allow filling of the bladder and to prevent urination, the one or more electrodes being electrically coupled to the pulse generator to receive the electrical waveforms from the pulse generator;
responsive to a readiness trigger being indicated to empty the bladder, applying, by the controller adjusting the operation of the pulse generator, a second electrical stimulation to the nervous system using the one or more electrodes to facilitate a voiding cycle of the bladder; and
responsive to completion of the voiding cycle, resume applying, by the controller readjusting the operation of the pulse generator, the first electrical stimulation to the nervous system using the one or more electrodes to again relax the bladder of the patient and to prevent urination.

2. The method of claim 1, wherein the first electrical stimulation includes applying 5-10 Hz stimulation to the pudendal nerve to relax the bladder, and the second electrical stimulation includes applying 5-10 kHz stimulation to the pudendal nerve to relax the external urethral sphincter (EUS) and applying 20-100 Hz stimulation to the pudendal nerve to contract the bladder.

3. The method of claim 1, wherein the first electrical stimulation includes applying 5-20 Hz stimulation to the sacral nerve to relax the bladder, and the second electrical stimulation includes applying 5-20 kHz stimulation to the EUS to relax the EUS and applying 20-100 Hz stimulation to the sacral nerve contract the bladder.

4. The method of claim 1, further comprising:
sending a notification to a remote-control device responsive to a voiding trigger being met; and
receiving the readiness trigger from the remote-control device confirming readiness of the patient to empty the bladder.

5. The method of claim 4, wherein the voiding trigger is indicated responsive to expiration of a timer.

6. The method of claim 4, wherein the voiding trigger is indicated responsive to a bladder sensor indicating a full condition of the bladder.

7. The method of claim 6, further comprising:
performing a calibration of the bladder sensor by measuring signals from the bladder sensor indicative of a level of fill of the bladder responsive to introducing one or more predefined amounts of liquid into the bladder;
receiving the signals from the bladder sensor; and
utilizing the calibration to determine that the signals from the bladder sensor indicate the full condition.

8. The method of claim 4, wherein the readiness trigger is indicated responsive to a signal from a urinal sensor indicating that a urinal is detected.

9. The method of claim 1, wherein the completion of the voiding cycle is indicated responsive to user input to a remote-control device.

10. The method of claim 1, wherein the completion of the voiding cycle is detected responsive to a urinal weight reaching a predefined threshold weight.

11. The method of claim 1, wherein the completion of the voiding cycle is detected responsive to a urinal fill level reaching a predefined threshold fill level.

12. The method of claim 1, wherein the completion of the voiding cycle is detected responsive to expiration of a predefined timeout period for urination.

13. The method of claim 1, further comprising:
recording aspects of a fill level of the bladder before and after the voiding cycle; and
computing an effectiveness of the voiding cycle as a ratio of the aspects of the fill level before the voiding divided by the same aspects of the fill level after the voiding.

14. The method of claim 1, further comprising:
recording, by the controller, times at which voiding cycles of the bladder are performed; and
responsive to the controller determining a change in frequency of the voiding cycles being greater than a first predefined threshold amount, switching from use of the one or more electrodes to use of one or more other electrodes.

15. The method of claim 14, wherein the first predefined threshold amount is indicative of a changed electrode lead position.

16. The method of claim 14, further comprising responsive to the controller determining the change in the frequency of the voiding cycles is less that than the first predefined threshold amount but greater than a second predefined threshold amount, adjusting aspects of the electrical waveforms being used by the controller to the voiding cycles.

17. The method of claim 16, wherein the second predefined threshold amount is indicative of scarring fibrosis at a nerve site of the one or more electrodes.

18. The method of claim 16, wherein the aspects of the electrical waveforms include one or more of frequency, amplitude, or pulse width.

19. The method of claim 16, further comprising utilizing a decision tree to identify which of the aspects of electrical waveforms are to be adjusted.

20. A system for selectively controlling urinary function, comprising:
a pulse generator connected to a nervous system of a patient by one or more electrodes configured to receive electrical waveforms from the pulse generator; and
a controller programmed to:
apply a first electrical stimulation from the pulse generator to the nervous system to relax a bladder of the patient to allow filling of the bladder and to prevent urination,
responsive to a readiness trigger being indicated to empty the bladder, apply a second electrical stimulation from the pulse generator to facilitate a voiding cycle of the bladder, and
responsive to completion of the voiding cycle, reapply the first electrical stimulation to again relax the bladder of the patient and to prevent urination.

21. The system of claim 20, wherein the one or more electrodes includes a first electrode connected to the pudendal nerve and a second electrode connected to the pudendal nerve, and the controller is further programmed to:
direct the pulse generator, as the first electrical stimulation, to apply 5-10 Hz stimulation to the pudendal nerve via the first electrode to relax the bladder; and
direct the pulse generator, as the second electrical stimulation, to apply 5-10 kHz stimulation to the pudendal nerve via the second electrode to relax the external urethral sphincter (EUS) and to apply 20-100 Hz stimulation to the pudendal nerve via the first electrode to contract the bladder.

22. The system of claim 20, wherein the one or more electrodes includes a first electrode connected to the sacral nerve and a second electrode connected to the EUS, and the controller is further programmed to:
direct the pulse generator, as the first electrical stimulation, to apply 5-20 Hz stimulation to the sacral nerve via the first electrode to relax the bladder; and
direct the pulse generator, as the second electrical stimulation, to apply 5-20 kHz stimulation to the EUS via the second electrode to relax the EUS and to apply 20-100 Hz stimulation to the sacral nerve via the first electrode to contract the bladder.

23. The system of claim 20, wherein the controller is further programmed to:
send a notification to a remote-control device, via a wireless transceiver, responsive to a voiding trigger being met; and
receive the readiness trigger from the remote-control device, via the wireless transceiver, responsive to the remote-control device confirming readiness of the patient to empty the bladder.

24. The system of claim 23, wherein the controller is further programmed to indicate the voiding trigger responsive to expiration of a timer.

25. The system of claim 23, further comprising a bladder sensor configured to provide a signal indicative of a fill level of the bladder to the controller, wherein the controller is further programmed to indicate the voiding trigger responsive to the bladder sensor indicating a full condition of the bladder.

26. The system of claim 25, wherein the controller is further programmed to:
perform a calibration of the bladder sensor by measuring signals from the bladder sensor indicative of the level of fill of the bladder responsive to introducing one or more predefined amounts of liquid into the bladder;
receive the signals from the bladder sensor; and
utilize the calibration to determine that the signals from the bladder sensor indicate the full condition.

27. The system of claim 23, wherein the controller is further programmed to indicate the readiness trigger responsive to a signal from a urinal sensor indicating that a urinal is detected.

28. The system of claim 20, wherein the controller is further programmed to indicate the completion of the voiding cycle responsive to user input to a remote-control device.

29. The system of claim 20, wherein the controller is further programmed to indicate the completion of the voiding cycle responsive to a urinal weight reaching a predefined threshold weight.

30. The system of claim 20, wherein the controller is further programmed to indicate the completion of the voiding cycle responsive to a urinal fill level reaching a predefined threshold fill level.

31. The system of claim 20, wherein the controller is further programmed to indicate the completion of the voiding cycle responsive to expiration of a predefined timeout period for urination.

32. The system of claim 20, wherein the controller is further programmed to:
record aspects of a fill level of the bladder before and after the voiding cycle; and
compute an effectiveness of the voiding cycle as a ratio of the aspects of the fill level before the voiding divided by the same aspects of the fill level after the voiding.

33. The system of claim 20, wherein the controller is further programmed to:
record, by the controller, times at which voiding cycles of the bladder are performed; and
responsive to the controller determining a change in frequency of the voiding cycles being greater than a first predefined threshold amount, switch from use of the one or more electrodes to use of one or more other electrodes.

34. The system of claim 33, wherein the first predefined threshold amount is indicative of a changed electrode lead position.

35. The system of claim 33, wherein the controller is further programmed to, responsive to the controller determining the change in the frequency of the voiding cycles is less that than the first predefined threshold amount but greater than a second predefined threshold amount, adjust aspects of the electrical waveforms being used by the controller to the voiding cycles.

36. The system of claim 35, wherein the second predefined threshold amount is indicative of scarring fibrosis at a nerve site of the one or more electrodes.

37. The system of claim 35, wherein the aspects of the electrical waveforms include one or more of frequency, amplitude, or pulse width.

38. The system of claim 35, further comprising utilizing a decision tree to identify which of the aspects of electrical waveforms are to be adjusted.
